(19) United States
(12) Patent Application Publication
(10) Pub. No.: US 2017/0160265 A1
(43) Pub. Date: Jun. 8, 2017

(54) COMPOSITIONS FOR DIRECT BREATH SAMPLING

(71) Applicant: TECHNION RESEARCH & DEVELOPMENT FOUNDATION LTD., Haifa (IL)

(72) Inventors: Hossam HAICK, Haifa (IL); Yoav BROZA, Haifa (IL)

(73) Assignee: Technion Research & Development Foundation Ltd., Haifa (IL)

(21) Appl. No.: 15/325,121

(22) PCT Filed: Jul. 20, 2015
(86) PCT No.: PCT/IL2015/050742

§ 371 Date: Jan. 10, 2017

Related U.S. Application Data
(60) Provisional application No. 62/026,739, filed on Jul. 21, 2014.

(51) Int. Cl. G01N 33/497; G01N 1/22

(52) U.S. Cl. CPC G01N 33/497; G01N 1/2214; G01F 1/00

(57) ABSTRACT

A composition, apparatuses and a methods for collecting and detecting compounds, including but not limited to volatile organic compounds, in a human breath sample are provided. In some embodiments, there is provided a glass-wool matrix and a sorbent material distributed throughout the glass-wool matrix.

### CROSS REFERENCE TO RELATED APPLICATIONS

This application claims the benefit of U.S. Provisional Patent Application No. 62/026,739, filed Jul. 21, 2014 and entitled “COMPOSITIONS FOR DIRECT BREATH SAMPLING”, the contents of which are incorporated herein by reference in their entirety.

### FIELD OF INVENTION

The present invention is directed to; inter alia, a device and method for direct breath sampling.

### BACKGROUND OF THE INVENTION

Breath analysis methodology is based on the collection and analysis of breath samples from human and/or animal subjects. Currently, methods for breath analysis sampling can be divided into two main options: i) direct breath into the sampling apparatus, and ii) indirect sampling using sampling bags or canisters. To avoid dilution or loss of sample, direct breath sampling is many times preferred. However, due to the high cost of the analyzing systems, direct breath sampling is not always possible. Therefore, there is a need for ex-situ sampling, wherein a sample is collected and optionally sent to a relevant data center without dilution or loss of breath compounds.

In order for this sampling to be efficient one would require a small, easy to use, cheap and long term storage solution. Sampling bags are easy to use but have a limited storage time and also present a substantial data lose due to condensation in the bag. Canisters are very efficient in storing samples but are very expensive and require a big storage space and heavy logistics.

The use of tubes filled with sorbent material(s) is a powerful solution as tubes are a relatively small and easy to use option. Currently, sorbent tubes are manufactured in different sizes according to the system used. Sorbent tubes can be stacked with different sorbent material (e.g., Tenax® TA, Carboxen and more) according to the target chemicals (e.g., volatile organic compounds) of interest. Normally, sorbent material is stacked in one, two or three beds and held by glass wool or glass frits on the ends of each sorbent material thus keeping the material in place. Sorbent amount/weight can change according to the material used and the purpose of use. This weight is proportional to the amount of chemicals that can be absorbed, i.e., more sorbent material more sorption place. Sorbent tubes are packed tightly with the sorbent material, and, therefore, are generally used with active sampling, i.e., using a pump or similar to achieve a flow of the interest gas/sample through the tube. Different tubes are applicable for gas volumes of few ml and up to tens of liters over timescales of minutes to hours. Therefore, in regards to breath sampling the protocol involves a two-step sampling: 1) breathing into a bag or canister/holder 2) actively pumping the breath from the collection apparatus (e.g., bag) to the sorbent tube. A great solution for overcoming this two-step procedure would be to allow direct sampling of breath into the sorbent tube. However, the rigid stacking of the sorbent material in the tube creates rather high resistance thus preventing one to blow directly into the tube.

Currently, there is no direct sampling into the sorbent tubes. There are few systems that allow sampling of breath into such sorbent tubes, but they have some drawbacks. Current sampling systems using sorbent tubes, e.g., the BCA system of Menssana Research Inc. and the EXP'AIR system of Ar2i company. Both systems are rather expensive (tens of thousands of dollars) and are big systems that require bench space and electric supply.

The BCA system is a long stainless steel (SS) tube (−90 cm long) with an external pump connected to the sorbent tube on the end of the SS tube. With breath taken from one end using a mouthpiece and the sampled tube is filled on the other end using the external pump system.

The EXP'AIR system is a big chest (80-90 cm long and 40 cm wide) wherein a pump is connected to a series of tubing and in parallel to the sorbent tube. In addition to its big dimensions and power consumption, the specific tubing that collects the breath to the sorbent tube cause heavy background noise in the sample, making the system non-efficient for the breath analysis.

The Bio-VOC Breath Sampler is a disposable device used, firstly, to collect a 100 ml sample of end-tidal air and then to transfer it to a sorbent tube. This system requires two-steps (to the chamber and then from the chamber to the tube) and suffers from extensive condensation and thus loss of VOCs.

There is a need for small, easy to use, cheap and long term sampling solutions for achieving direct breath sampling procedures.

The foregoing examples of the related art and limitations related therewith are intended to be illustrative and not exclusive. Other limitations of the related art will become apparent to those of skill in the art upon a reading of the specification and a study of the figures.

### SUMMARY OF THE INVENTION

The present invention provides, in some embodiments, a composition of glass-wool and sorbent material suitable for direct breath sampling. In additional embodiments, there is provided an apparatus comprising said composition, a method for its preparation and methods for sampling breath comprising molecules of interest, e.g., Volatile Organic Compounds (VOCs).

In one aspect, the present invention provides an apparatus comprising a body comprising an inlet, an outlet and a cavity between the inlet and the outlet, the cavity comprising a glass-wool matrix and a sorbent material distributed throughout the glass-wool matrix.

In some embodiments, said glass-wool has a weight of 10 to 150 milligrams (mg). In some embodiments, the sorbent material has a weight of 10 to 500 mg. In another embodiment, the ratio between the glass-wool and the sorbent material is of 1:1-1.5:1. In another embodiment, the ratio between the glass-wool and the sorbent material is of 1:1-1:5. In another embodiment, the apparatus comprises a substantially homogeneous matrix of the glass-wool and sorbent material.

In some embodiments, the sorbent material is selected from the group consisting of: Tenax®, Carbotrap®, Carboxen®, Carbosieve®, Anasorb ®, Carbograph®, Chromosorb®, Carbopack®, Amberlite® XAD, Supelpak®-2, HMP, carbon nanotubes, glass bead, polymers, molecular sieves, activated carbons, Coconut charcoal, HayeSep®, ceramics, aluminas, silicas, silica gels, molecular sieve carbon, molecular sieve zeolites, silicalite, and combinations thereof.

In some embodiments, the glass-wool includes at least one of borosilicate glass wool, quartz glass wool, and glass fiber.

In another embodiment, the body of the apparatus defines a conduit between the inlet and the outlet. In some embodiments, the body is configured for flowing of VOCs therethrough. In some embodiments, the body is a thermal desorption tubes. In some embodiments, the inlet and the outlet of the body is a sampling inlet and a sampling outlet, respectively. In another embodiment, the sampling inlet is configured to be operably connected to a nozzle.

In another embodiment, the apparatus further comprising a flow meter (such as a built-in flow-meter).

In another aspect, the present invention provides a method of sampling compounds in a breath sample of a subject in need thereof, the method comprising: providing the apparatus described herein; and exhaling into the apparatus.

In another embodiment, the compounds are VOCs.

In another embodiment, the exhaling has a volume flow rate in the range of 1 milliliters/minute-500 milliliters/minute.

In another embodiment, the subject is a mammal.

In another aspect, the present invention provides a composition comprising a glass-wool matrix and a sorbent material for use in sampling compounds in a breath sample of a subject.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

Exemplary embodiments are illustrated in referenced figures. Dimensions of components and features shown in the figures are generally chosen for convenience and clarity of presentation and are not necessarily shown to scale. The figures are listed below.

FIG. 1A is a cross sectional view of a body of an apparatus in accordance with an embodiment;

FIG. 1B is a cross sectional view of an exemplary implementation of the apparatus of FIG. 1A in accordance with an embodiment;

### DETAILED DESCRIPTION OF THE INVENTION

The present invention provides, in some embodiments, a composition of glass-wool and sorbent material and a device/apparatus comprising the composition. In some embodiments, the composition and device/apparatus are useful for direct breath sampling. Additional embodiments of the invention relate to a kit comprising the breath sampling device/apparatus, a method for its preparation and methods for breath sampling.

The present invention is based, in part, on finding the glass-wool can be used, not only as an end plug for holding a sorbent material, but rather to form a matrix incorporating sorbent material there within. As exemplified herein, a matrix of glass-wool incorporated with sorbent material enables the direct sampling of breath of a subject.

In some embodiments, the composition or matrix of sorbent material and glass-wool has low resistance (e.g., compared to commonly used sampling devices/apparatuses or sorbent tubes), thereby permitting direct sampling of breath Volatile Organic Compounds (VOCs). In some embodiments, the low resistance is below 30 millimeter of mercury (mmHg), below 20 mmHg, below 15 mmHg, below 10 mmHg. Each possibility represents a separate embodiment of the invention.

In some embodiments, the composition of glass-wool and sorbent material forms a substantially homogenous matrix. In some embodiments, incorporation of the sorbent in the glass wool matrix is by methods known to one skilled in the art.

In another embodiment, the ratio between the glass-wool and the sorbent material is of 1:1-5:1, or any ratio in between these illustrative ratios. In another embodiment, the ratio between the glass-wool and the sorbent material is of 1:1-4:1. In another embodiment, the ratio between the glass-wool and the sorbent material is of 1:1-3:1. In another embodiment, the ratio between the glass-wool and the sorbent material is of 1:1-2.5:1. In another embodiment, the ratio between the glass-wool and the sorbent material is of 1:1-2:1. In another embodiment, the ratio between the glass-wool and the sorbent material is of 1:1-1.5:1. Each possibility represents a separate embodiment of the invention.

In another embodiment, the ratio between the glass-wool and the sorbent material is of 1:1-1:5, or any ratio in between these illustrative ratios. In another embodiment, the ratio between the glass-wool and the sorbent material is of 1:1-1:4. In another embodiment, the ratio between the glass-wool and the sorbent material is of 1:1-1:3.5. In another embodiment, the ratio between the glass-wool and the sorbent material is of 1:1-1:2. In another embodiment, the ratio between the glass-wool and the sorbent material is of 1:1-1:1.5. Each possibility represents a separate embodiment of the invention.

In some embodiments, the matrix of glass-wool has a weight of at most 500 milligrams (mg), at most 400 mg, at most 300 mg, at most 200 mg, at most 175 mg, at most 150 mg, at most 140 mg, at most 130 mg, at most 120 mg, at most 110 mg, at most 100 mg, at most 90 mg, at most 80 mg, at most 70 mg, at most 60 mg, at most 50 mg, at most 40 mg or at most 50 mg. Each possibility represents a separate embodiment of the invention. In some embodiments, the glass-wool has a weight of at least 10 mg, at least 20 mg, at least 30 mg, at least 40, at least 50 mg, at least 60 mg, at least 70, at least 80 mg, at least 90 mg, at least 100 mg, at least 110 mg, at least 120 mg, at least 130, at least 140 or at least 150 mg. Each possibility represents a separate embodiment of the invention.

In some embodiments, the sorbent material has a weight of at most 500 mg, at most 400 mg, at most 300 mg, at most 200 mg, at most 175 mg, at most 150 mg, at most 140 mg, at most 130 mg, at most 120 mg, at most 110 mg, at most 100 mg, at most 90, at most 80 mg, at most 70 mg, at most 60 mg, at most 50 mg, at most 40 mg, at most 30 mg, at most 20 mg or at most 10 mg. Each possibility represents a separate embodiment of the invention. In some embodiments, the sorbent material has a weight of at least 10 mg, at least 20 mg, at least 30 mg, at least 40 mg, at least 45 mg, at least 50 mg, at least 60 mg, at least 70 mg, at least 80 mg, at least 90 mg, at least 100 mg, at least 110 mg, at least 120 mg, at least 130 mg, at least 140 mg, at least 150 mg, at least 175 mg, at least 200 mg, at least 300 mg, at least 400 mg or at least 500 mg. Each possibility represents a separate embodiment of the invention.

In some embodiments, the sorbent material is a porous material (e.g., Poly(2,6-diphenyl-p-phenylene oxide). In another embodiment, the matrix has a target porosity of more than 0.70, more than 0.80, more than 0.85 or more than 0.90. In another embodiment, the matrix has a target density of less than 0.5 gram/cubic centimeters (gram/cc), less than 0.4 gram/cc or less than 0.3 gram/cc. Each possibility or any value in between these values represents a separate embodiment of the invention.

In some embodiments, the sorbent material is a non-porous material (e.g., graphitized carbon black (GCB) adsorbents). In certain embodiments, one or more of the sorbent material types used in the sorbent apparatus described herein may be based on, or include, a graphitized carbon black (GCB), a carbon molecular sieve, or combinations thereof. In some examples, the sorbent material may be based on a mixture of graphitized carbon blacks of different strengths, graphite, carbon molecular sieves, polymer resins, an oxide, fused silica beads, glass, quartz, charcoal, porous polymers, amisorbs or other materials. In certain embodiments, the different sorbent material in the sorbent apparatus may have a different chemical composition, e.g., each may include or be a different carbon black. In some examples, the sorbent material may be a derivatized form, e.g., a derivatized carbon black.

In some examples, the sorbent material can be a graphitized carbon black such as, for example, Carbotrap™ B sorbent or Carbopack™ B sorbent, Carbotrap™ Z sorbent or Carbopack™ Z sorbent, Carbotrap™ C sorbent or Carbopack™ C sorbent, Carbotrap™ X sorbent or Carbopack™ X sorbent, Carbotrap™ Y sorbent or Carbopack™ Y sorbent, Carbotrap™ F sorbent or Carbopack™ F sorbent, any one or more of which may be used in its commercial form (available commercially from Supelco or Sigma-Aldrich) or may be graphitized according to known protocols. In other examples, the sorbent material can be carbon molecular sieves such as Carboxen™ 1000 sorbent, Carboxen™ 1003 sorbent, or Carboxen™-1016 sorbent, any one or more of which may be used in its commercial form (available commercially from Supelco or Sigma-Aldrich) or may be optimized according to known protocols.

Additional none limiting examples of sorbent materials include Tenax® (2,6-diphenylene-oxide polymer), Anasorb®, Chromosorb®, Amberlite® XAD, Supelpak®-2, HayeSep®, HMP, carbon nanotubes, glass bead, polymers, molecular sieves, activated carbons, coconut charcoal, ceramics, aluminas, silicas, silica gels, molecular sieve carbon, molecular sieve zeolites, silicalite, and combinations thereof.

Silica gel, as used herein, refers to an amorphous form of silicon dioxide, which is synthetically produced in the form of hard irregular granules or beads. A microporous structure of interlocking cavities provides a very high surface area (800 square meters per gram). This unique structure renders the silica gel as a high capacity desiccant. Water molecules adhere to the surface of the silica gel due to its low vapor pressure as compared to the surrounding air. When pressure equilibrium is reached, the adsorption ceases. Thus, the higher the humidity of the surrounding air, the larger the amount of water that is adsorbed before equilibrium is reached. Silica gel is advantageous as a drying substance since the process of drying does not require any chemical reaction and it does not produce any by products or side effects.

Activated carbon, as used herein, refers to a sorbent formed by processing charcoal to an extremely porous carbon substance. Due to its high degree of microporosity, the activated carbon possesses a very large surface area available for chemical reactions. Sufficient activation may be obtained solely from the high surface area, though further chemical treatments often enhance the adsorbing properties of the material.

Desiccant molecular sieves, as used herein, refers to synthetically, highly porous crystalline metal-alumino silicates. They are classified by the many internal cavities of precise diameters, namely, 3 angstroms (Å), 4 Å, 5 Å, and 10 Å. Adsorption occurs only when molecules to be adsorbed have smaller diameters than the cavity openings.

The particular type and amount of sorbent materials may be selected depending on the particular VOC to be adsorbed as well as flow rates, flow volumes and concentration levels.

In some embodiments where plurality of sorbent materials are used, a first sorbent material may be included in a larger amount that a second sorbent materials. For example, where a sample is suspected of having a large concentration of a particular analyte, the sorbent material effective to adsorb and desorb that analyte may be present in a larger amount/volume to provide for increased loading of that analyte. In certain examples, the sorbent materials can each be present at substantially the same weight ratio, e.g., 1:1. In other examples, the different sorbent materials can independently be present in weight ratios ranging from 3:1, 2.5:1, 2:1, 1.5:1, 1.1:1, 0.9:1, 0.8:1, 0.7:1, 0.6:1, 0.5:1, 0.4:1, 0.3:1, 0.2:1, 0.1:1 or any ratio in between these illustrative ratios. Additional suitable amounts of the sorbent materials will be readily selected by the person of ordinary skill in the art.

In certain examples, the mesh size or range of the sorbent can vary depending on the particular material selected. In some examples, the mesh size can range from 20 to about 100, more particular from about 20-80, 30-70 or 40-60. In other examples, the mesh size range may be from about 20-40, 40-60, 60-80 or 80-100 depending on the material used in the sorbent apparatus. Other suitable mesh sizes will be readily selected by the person of ordinary skill in the art.

In some embodiments, the glass-wool includes at least one of borosilicate glass wool, quartz glass wool, and glass fiber.

In some embodiments, the apparatus is devoid of glass-wool end plugs. In some embodiments, the apparatus may further include glass-wool as an end plug to hold the glass wool-sorbent material composition. In the embodiments, the end plug glass-wool does not substantially raise the resistance of the composition (e.g., that the apparatus may still be used for direct breath sampling). As used herein an “end plug glass-wool that does not substantially raise the resistance of the composition” is a glass wool having a width of about 3 to 5 mm, with porosity of more than 0.90, and total density range of 0.10 to 0.90 grams/cc.

According to some embodiments of the invention, use of the composition described herein results in minimal loss or dilution of VOCs found in the original breath sample. In some embodiments, less than 10%, less than 9%, less than 8%, less than 7%, less than 6%, less than 5%, less than 4%, less than 3%, less than 2% or less than 1% VOCs are loss (e.g., not adsorbed) using the composition of the invention.

In some embodiments, the present invention provides an apparatus comprising a body comprising an inlet, an outlet and a cavity between the inlet and the outlet, the cavity comprising the composition of glass-wool and at least one sorbent material. In another embodiment, the body of the apparatus defines a conduit between the inlet and the outlet. In some embodiments, the body is configured for flowing of VOCs there through and collecting (i.e., sampling) the VOCs. In some embodiments, the body is a sorbent tube. In some embodiment, the sorbent tube may be made from any suitable one or more materials known in the art. In some embodiments the sorbent tube is made of glass. In some embodiments, the inlet and the outlet of the body is a sampling inlet and a sampling outlet, respectively. In another embodiment, the sampling inlet is configured to be operably connected to a nozzle and/or a mouthpiece.

In some embodiments, breathing directly into an apparatus comprising the composition, includes breathing through a mouthpiece or nozzle operably-connected to the apparatus described herein. In another embodiment, the mouthpiece may be connected to the tubular device using tubing adaptors, including but not limited to Union Connector Let-Lok® Tube Fitting, ¼″ Nut, replaceable ¼″ PTFE ferrule, Port Connector.

In additional embodiments, the apparatus or system comprising the apparatus further includes a breath flow meter.

Typically, normal breath includes both alveolar breath and airway breath. Alveolar breath is known in the art as that portion of the breath which has originated in the alveoli (“air sacs”) of the lungs, having been drawn there by inhalation for gaseous interchange with capillary blood. Airway breath, which is also known as “dead space” breath, is that portion of the breath which has originated in the bronchial tubes, the trachea, pharynx and mouth and nasal cavities, and comprises air in a given inhalation which has not reached the alveoli, and which therefore has not been involved in any gaseous interchange within the body. For efficient sampling, a breath sampling apparatus can control the breath sampling by collecting only the alveolar breath component, not the dead space.

In some embodiments, the apparatus or system comprising the apparatus further includes a dead space bag. Dead space bag may be made from any suitable materials known in the art.

In another embodiment, the apparatus or system does not require electric power or a pumping unit.

According to some embodiments of the invention, said low resistance is further useful for sampling particularly low volume flow. In some embodiments, low volume flow may be produced by exhaling air for sampling. In some embodiments, low volume flow includes rates less than 1 milliliters/minute. In some embodiments, low volume flow includes rates ranging from 1 milliliters/minute-500 milliliters/minute. In some embodiments, the invention further permits low-potency sampling including but not limited to infants, kids and elderly subjects, subject having respiratory diseases or disorders (e.g., with breathing difficulties), as well as animals.

In some embodiments, the invention further provides a method of sampling compounds in a breath sample of a subject in need thereof, the method comprises: providing an apparatus comprising a body comprising an inlet, an outlet and a cavity between the inlet and the outlet, the cavity comprising a glass-wool matrix and a sorbent material distributed throughout the glass-wool matrix; and exhaling into the apparatus.

In some embodiments, methods of breath sampling of the invention are used for, or include a step of, transferring the sample to analytical or sensor based analysis systems. None limiting uses of the methods of the invention include clinical, industrial and security uses.

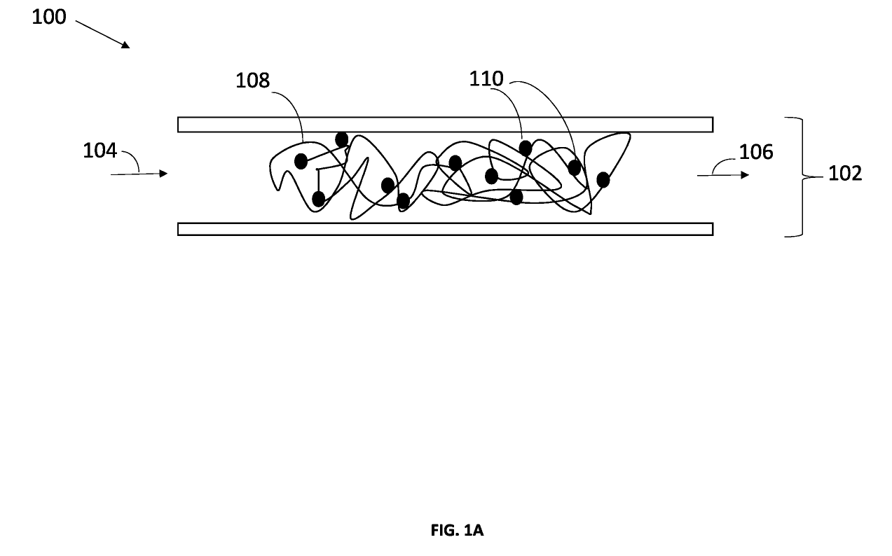

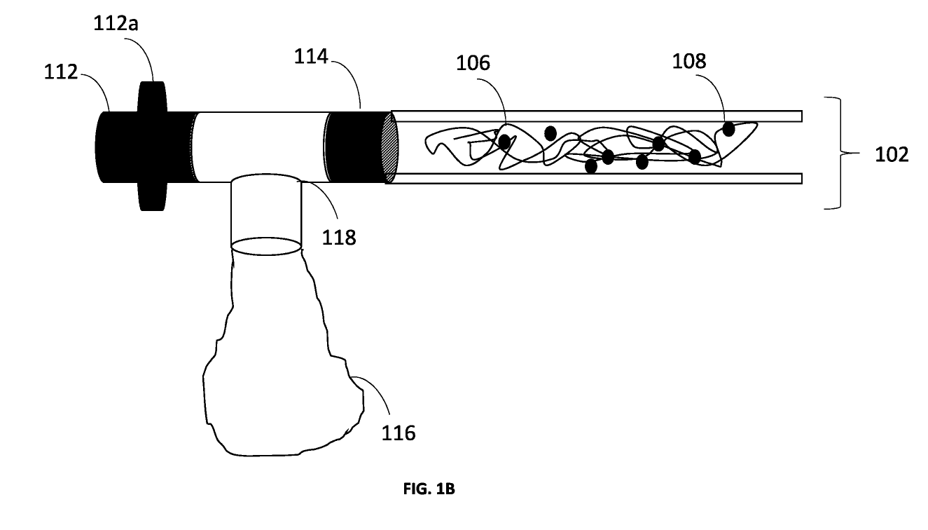

Reference is now made to FIG. 1A which shows a cross sectional view of an apparatus 100. Apparatus 100 includes a tube (e.g., thermal desorption tube) 102 having an inlet 104 and an outlet 106 facilitating a flow of gas/sample through tube 102. Comprised within tube 102 is a glass-wool matrix 108 and a sorbent material 110 distributed throughout the glass-wool matrix.

Reference is now made to FIG. 1B which shows a cross sectional view of an exemplary implementation of apparatus 100 that may be used for breath sampling. Tube 102 is connected to a mouthpiece 112 via an adaptor 114. Optionally mouthpiece 112 may include a filter 112a to prevent inlet of bacteria and/or viruses through tube 102. In a non-limiting example adaptor 114 is made of stainless steel (SS). Optionally, a dead space bag 116 is connected via a T-valve 118 located between mouthpiece 112 and adaptor 114.

As used herein and in the appended claims the singular forms “a”, “an,” and “the” include plural references unless the content clearly dictates otherwise. Thus, for example, reference to “an organic coating” includes a plurality of such organic coatings and equivalents thereof known to those skilled in the art, and so forth. It should be noted that the term “and” or the term “or” is generally employed in its sense including “and/or” unless the content clearly dictates otherwise.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. An apparatus comprising a body comprising an inlet, an outlet and a cavity between the inlet and the outlet, the cavity comprising a glass-wool matrix and a sorbent material distributed throughout the glass-wool matrix.

2. The apparatus of claim 1, comprising 10 to 150 milligrams weight of glass-wool.

3. The apparatus of claim 1, comprising 10 to 500 milligrams by weight of sorbent material.

4. The apparatus of claim 1, comprising a ratio of 1:1-1.5:1 between said glass-wool and said sorbent material.

5. The apparatus of claim 1, comprising a ratio of 1:1-1:5 between said glass-wool and said sorbent material.

6. The apparatus of claim 1, comprising a substantially homogeneous matrix of said glass-wool and sorbent material.

7. The apparatus of claim 1, wherein said sorbent material is selected from the group consisting of: Tenax®, Carbotrap®, Carboxen®, Carbosieve®, Anasorb®, Carbograph®, Chromosorb®, Carbopack®, Amberlite® XAD, Supelpak®-2, HMP, carbon nanotubes, glass bead, polymers, molecular sieves, activated carbons, Coconut charcoal, HayeSep®, ceramics, aluminas, silicas, silica gels, molecular sieve carbon, molecular sieve zeolites, silicalite, and combinations thereof.

8. The apparatus of claim 1 wherein said glass-wool includes at least one of borosilicate glass wool, quartz glass wool, and glass fiber.

9. The apparatus of claim 1 wherein said body defines a conduit between said inlet and said outlet configured for flowing of volatile organic compounds (VOCs) there through.

10. The apparatus of claim 1, wherein said body is a thermal desorption tubes.

11. The apparatus of claim 1, wherein said inlet and said outlet of said body is a sampling inlet and a sampling outlet, respectively.

12. The apparatus of claim 11, wherein said sampling inlet is configure to be operably connected to a nozzle.

13. The apparatus of claim 1, further comprising a flow meter.

14. A method of sampling compounds in a breath sample of a subject in need thereof, the method comprising:
(a) providing an apparatus comprising a body comprising an inlet, an outlet and a cavity between the inlet and the outlet, the cavity comprising a glass-wool matrix and a sorbent material distributed throughout the glass-wool matrix; and
(b) exhaling into said apparatus.

15. The method of claim 14, wherein said compound is volatile organic compounds (VOC).

16. The method of claim 14, wherein said exhaling has a volume flow rate in the range of 1 milliliters/minute-500 milliliters/minute.

17. The method of claim 14, wherein said subject is a mammal.
